(19) United States
(12) Patent Application Publication
(10) Pub. No.: US 2009/0248157 A1
(43) Pub. Date: Oct. 1, 2009

(54) Biocompatible Substrate and Method for Manufacture and Use Thereof

(76) Inventors: Matthew Dalby, Glasgow Scotland (GB); Nikolaj Gadegaard, Glasgow Scotland (GB)

Correspondence Address: NIXON & VANDERHYE, PC, 901 NORTH GLEBE ROAD, 11TH FLOOR, ARLINGTON, VA, 22203, US

(21) Appl. No.: 12/085,182

(22) PCT Filed: Nov. 17, 2006
(86) PCT No.: PCT/GB2006/004307

§ 371 Date: May 19, 2008

(51) Int. Cl. A61F 2/28; C12M 1/00; C12N 5/00

(52) U.S. Cl. 623/16.11; 435/289.1; 435/377

(57) ABSTRACT

A biocompatible substrate for cell adhesion, differentiation, culture and/or growth, has an arrangement of topographical features arrayed in a pattern based on a notional symmetrical lattice in which the distance between nearest neighbor notional lattice points is C and is between 10 nm and 10 μm. The topographical features are locally mis-ordered such that the centre of each topographical feature is a distance of up to one half of C from its respective notional lattice point.

The present invention relates to biocompatible substrates, uses thereof, and methods for their manufacture.

Particularly, but not exclusively, the invention relates to biocompatible substrates that are useful as implant materials, such a bone implant materials.

References noted in abbreviated form in the body of the text are set out in full in the section at the end of the description headed “References”. The content of each reference is hereby incorporated by reference.

Conventionally, inert materials are used as surgical implants, the goal being simply to avoid implant rejection.

When considering, for example, a normal load bearing orthopaedic implant, this will have a metal stem fixed by polymethylmethacrylate (PMMA) cement. Charnley and Smith developed this type of implant in the 1960's (Charnley, Br Med J 1960 and Charnley, J Bone Joint Surg Br 1960). However, implants and materials are not particularly new to medicine; metals have been used for over 2000 years in, for example, dental restorations. Recently PMMA has come into use, which was noted to be tolerated when shards from shattered cockpits entered the eyes of pilots in the 1940s (France et al, 2000).

A major problem is that these ‘inert’ (although it is noted that no material is truly inert) materials are encapsulated by the body. For a load bearing prosthesis the formation of a soft capsule rather than direct bone integration leads to micromotion that is exacerbated by the modulus mismatch between the hard material (e.g. metal) and the softer bone (Freeman et al 1982). The difference in modulus leads to stress shielding of the supported bone leading to bone necrosis through lack of loading (Gefen 2002). For non-load bearing implants e.g. maxillofacial plates, the encapsulation results in low-quality repair.

It is known that the surface of the implant member can have an effect on the body's response to the implant. Even surface features at the nanoscale can affect cell response to materials. Fibroblasts have been shown to respond to surface features down to just 10 nm with filopodial sensing (Dalby et al 2004) Other responses include changes in morphology (Dalby et al, Biomaterials 2002), adhesion (Gallagher 2002), motility (Berry 2004), proliferation (Dalby et al, Tissue Eng 2002), endocytotic activity (Dalby et al, Exp Cell Res 2004) and gene regulation (Dalby et al, IEEE Transactions on Nanobioscience 2002) of a large number of cell types including fibroblasts (Dalby et al, Tissue Eng 2002, and Dalby et al, Exp Cell Res 2002), osteoblasts (Price et al, 2003), osteoclasts (Webster et al 2001), endothelial (Dalby et al, Biomaterials 2002), smooth muscle (Thapa et al 2003), epithelial (Andersson et al, Biomaterials 2003, and Andersson et al, IEEE Transactions on Nanobioscience 2003) and epitenon cells (Gallagher 2002).

The alteration of cellular function at nanostructured interfaces may result from direct influence on cellular responses or may result from an altered extracellular layer matrix deposited on the surface. Nanoscale topography has been shown to alter the functional behaviour of both adhesive (Sutherland et al, 2001) and connective tissue proteins (Denis et al, 2002).

Through the drive for miniaturisation lead by the microelectronics engineering there have been significant advancements in lithographic techniques. This mainly includes the shift from photolithography to electron-beam lithography (Gadegaard et al 2003, and Wilkinson et al 2002), which can give resolution down to 10 nm. Electron-beam lithography in particular has been shown to be suitable for forming nanoscale cues for the investigation of cellular responses (Curtis et al 2001; Gadegaard et al 2003; Cumming et al 1996; Vieu et al 2000). The inventors have realised that it would be particularly desirable to elicit specific cell responses using spatial cues provided by manufactured materials. In order to drive specific responses, such materials may stimulate the cells using physics (forces and interactions), chemistry or shape (topography).

In a general aspect, the present inventors have developed materials that are capable of influencing stem cell differentiation by providing a degree of mis-order to the symmetry of a nanoscale topography. In one particularly preferred aspect, the materials are capable of influencing mesenchymal stem cell differentiation into bone forming osteoblasts rather than into capsule forming fibroblasts. In another preferred aspect, the materials are capable of promoting osteoprogenitor cell differentiation into osteoblasts, which remain adhered to the substrate.

In a first preferred aspect, the present invention provides a biocompatible substrate for cell adhesion, differentiation, culture and/or growth, the substrate having an arrangement of topographical features arrayed in a pattern based on a notional symmetrical lattice in which the distance between nearest neighbour notional lattice points is C and is between 10 nm and 10 μm, and wherein the topographical features are locally mis-ordered such that the centre of each topographical feature is a distance of up to one half of C from its respective notional lattice point.

Using the invention, the present inventors have shown that the mis-order applied to the topographical features can have an unexpected beneficial effect on cell adhesion, differentiation, culture and/or growth.

Preferred and/or optional features will now be set out. These are applicable singly or in any combination, unless the context demands otherwise.

Preferably, the topographical features of the biocompatible substrate are recesses into and/or protrusions from the surface of the substrate. In particular, the topographical features may include pits. Additionally or alternatively, the topographical features may include upstanding pillars.

Preferably, C is at least 20 nm, at least 30 nm, at least 40 nm, at least 50 nm, at least 60 nm, at least 70 nm, at least 80 nm, at least 90 nm, at least 100 nm, at least 110 nm, at least 120 nm, at least 130 nm, at least 140 nm, at least 150 nm, at least 160 nm, at least 170 nm, at least 180 nm, at least 190 nm, at least 200 nm, at least 210 nm, at least 220 nm, at least 230 nm, at least 240 nm, at least 250 nm, at least 260 nm, at least 270 nm, at least 280 nm, at least 290 nm or about 300 nm.

Preferably, C is at most 9 μm, at most 8 μm, at most 7 μm, at most 6 μm, at most 5 μm, at most 4 μm, at most 3 μm, at most 2 μm, at most 1 μm, at most 900 nm, at most 800 nm, at most 700 nm, at most 600 nm, at most 500 nm, at most 400 nm.

The most preferred range for C is between 30 nm and 3 μm.

Preferably, the height or depth (e.g. the average height or depth) of the topographical features is at least 5%, more preferably at least 10%, of C from the remainder of the surface of the substrate. For example, the height or depth of the topographical features may be at least 10 nm.

Preferably, each topographical feature has the same shape. The topographical features may be cylindrical pits or pillars, cuboid pits or pillars, hemi-spherical pits or pillars, part-spherical pits or pillars, or another regular shape.

Preferably, the diameter of the topographical features is at least 10%, more preferably at least 20%, at least 30%, at least 40% or at least 50%, of C. For example, the diameter of the topographical features may be at least 20 nm.

Preferably, the centre of each topographical feature is at most 45%, more preferably at most 40%, at most 35%, at most one third, at most 30%, at most 25%, at most 20%, at most 15%, at most 10% or at most 5%, of C from its respective notional lattice point.

Preferably, for at least 50% of the topographical features, the centre of each topographical feature is between one tenth and one quarter of C from its respective notional lattice point. More preferably, at least 60%, at least 70%, at least 80% or at least 90% of the topographical features satisfy this criterion. The lower limit for the distance of the centre of each topographical feature from its respective notional lattice point is preferably at least 12% of C, at least 14% of C or at least 16% of C. The upper limit for the distance of the centre of each topographical feature from its respective notional lattice point is preferably at most 22% of C, at most 20% of C or at least 18% of C.

The nature of the symmetry on which the notional lattice is based may be selected from a parallelogram lattice, a rectangular lattice, a square lattice, a rhombic lattice, a trigonal lattice and a hexagonal lattice. Preferably, the notional lattice is either a rectangular lattice or a square lattice.

The substrate comprises a biocompatible material. Of particular interest here are polycarbonate and polymethylmethacrylate. However, other biocompatible polymers may be used. Furthermore, other biocompatible materials such as metals and ceramics may also be used. Additionally or alternatively, the substrate may be formed of a biocompatible composite material, for example, in which a surface layer or layers is formed of one of the biocompatible materials mentioned above. In the case of ceramics, it is preferred to cast and sinter the ceramics rather than perform an embossing step, which is a preferred route for polymer materials.

In a second preferred aspect, the invention provides a method of manufacturing a biocompatible substrate according to the first aspect, including the steps of designing the notional symmetrical lattice, applying a degree of mis-order to the notional symmetrical lattice by requiring that the centre of each topographical feature is up to one third of C from its respective notional lattice point, thereby designing a mis-ordered lattice, and manufacturing the substrate according to the mis-ordered lattice.

Preferred and/or optional features set out above may be applied in any combination to the second aspect of the invention.

Preferably, the degree of mis-order is applied to each notional lattice point by a calculation step in which a random number is generated and used to provide one or more displacement amounts to said notional lattice point. For example, for each lattice point of a rectangular or square lattice, a random displacement along one axis may be applied, followed by a random displacement along an orthogonal axis. For a non-orthogonal lattice (e.g. a parallelogram lattice, hexagonal lattice or trigonal lattice), these random displacements may be made along axes of the lattice, or along orthogonal axes. Typically, the random number generated is operated on using a multiplier, that multiplier corresponding to the fraction of C corresponding to the desired maximum mis-order of the array of topographical features.

Preferably, the method comprises the step of forming an array of topographical features using electron beam lithography. This array may be formed on the surface of a master substrate. The master substrate need not itself be a biocompatible substrate suitable for implantation into the human or animal body.

The master substrate may be used to create an intermediate substrate. For example, the intermediate substrate may be formed to provide the “negative” topographical features to those of the master substrate. The intermediate substrate may then be used to create the biocompatible substrate, e.g. by pressing or imprinting of the biocompatible substrate with the intermediate substrate. Alternatively, the biocompatible substrate may be created directly from the master substrate, if the “negative” of the topographical features of the master substrate is what is required for the biocompatible substrate. So, for example, if a biocompatible substrate is required having an array of nano-pillars, and the procedure for creating the master substrate provides a corresponding array of nano-pits, then the biocompatible substrate can be imprinted (or embossed) by the master substrate to provide the necessary topography.

Alternatively, the biocompatible substrate may be manufactured by injection moulding. Injection moulding has a significant advantage over embossing in that it is more suited to forming a substrate having a curved and/or non-uniform surface.

Preferably, the biocompatible substrate provides a means for assisting stem cell differentiation towards a preferred cell function. Most preferably, the biocompatible substrate provides a cue for preferential stem cell differentiation for osteoprogenitor cells, or for the progenitor cells of these cells.

In certain circumstances, it is preferred to provide an implant element for bone surgery, the implant comprising a biocompatible substrate according to the first aspect and having bone tissue cultured on the substrate. For certain types of bone surgery, it can be of assistance to apply one or more such implant elements at a bone trauma site in the patient via impaction grafting. For example, multiple implant elements (in the form of fragments) may be applied in this way. Such preparation of the bone trauma site can assist in the support of a main bone implant at that site.

In a third preferred aspect, the present invention provides a bone repair prosthesis (e.g. for hip, knee or maxillofacial repair) having a surface with an arrangement of topographical features arrayed in a pattern based on a notional symmetrical lattice in which the distance between nearest neighbour notional lattice points is C and is between 10 nm and 10 μm, and wherein the topographical features are locally mis-ordered such that the centre of each topographical feature is a distance of up to one half of C from its respective notional lattice point.

In a fourth preferred aspect, the present invention provides a biocompatible substrate according to the first aspect, for use in a method for treatment of the human or animal body by surgery or therapy.

In a fifth preferred aspect, the present invention provides a biocompatible substrate according to the first aspect, for use in bone implant surgery. For example, the bone implant surgery may include hip, knee or maxillofacial repair.

In a sixth preferred aspect, the present invention provides a use of a biocompatible substrate according to the first aspect in the manufacture of an implant member for the treatment of a condition requiring bone construction, reconstruction or repair.

In a seventh preferred aspect, the present invention provides a use of a biocompatible substrate according to the first aspect in bone implant surgery.

Specific embodiments of the invention will now be described, by way of example, with reference to the accompanying drawings, in which:

FIG. 1 shows a schematic plan view of a nanotopography according to an embodiment of the invention.

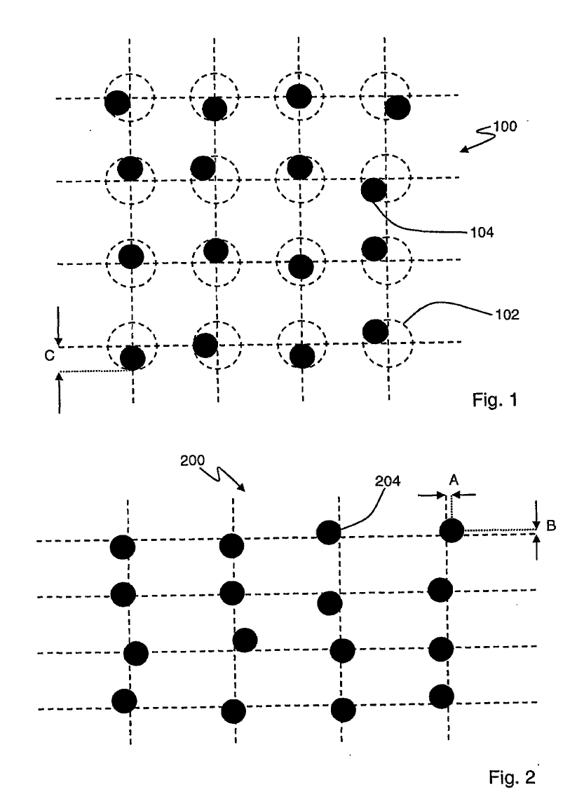

FIG. 2 shows a schematic plan view of a nanotopography according to an embodiment of the invention.

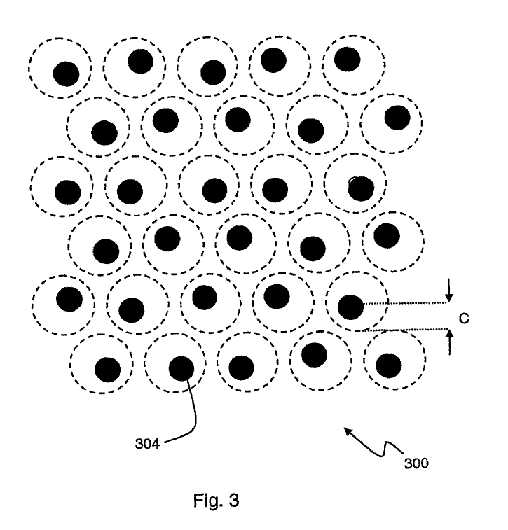

FIG. 3 shows a schematic plan view of a nanotopography according to an embodiment of the invention.

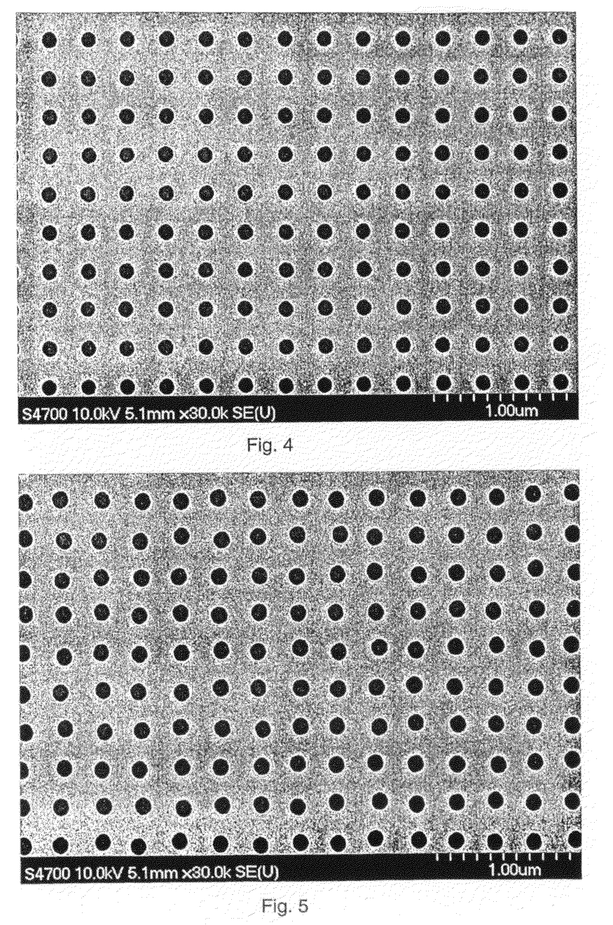

FIGS. 4-6 show SEM micrographs of different nanotopographies.

FIG. 7 shows SEM images and FFT images of the different nanotopographies of FIGS. 4-6.

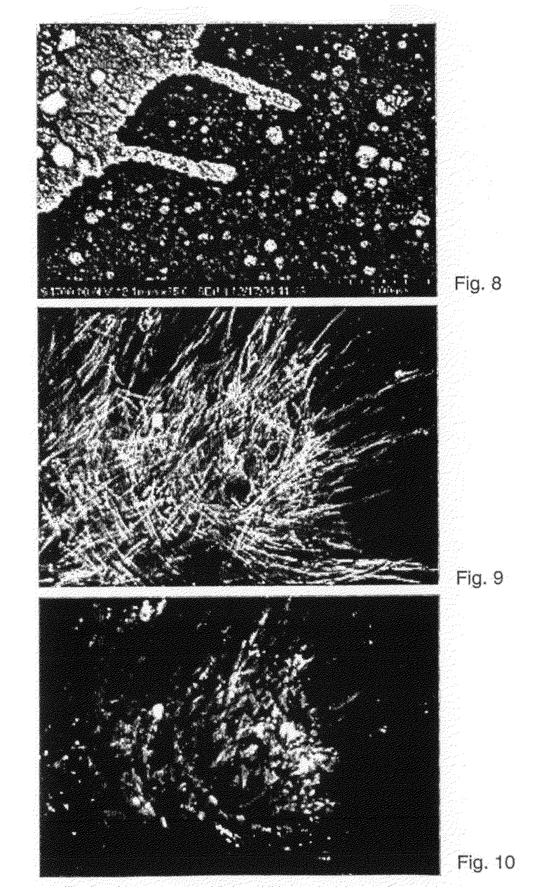

FIG. 8 shows an SEM image of filopodia of osteoprogenitor cells cultured on a substrate having orthogonal pits.

FIG. 9 shows actin staining of the substrate of FIG. 8.

FIG. 10 shows OPN staining of the substrate of FIG. 8.

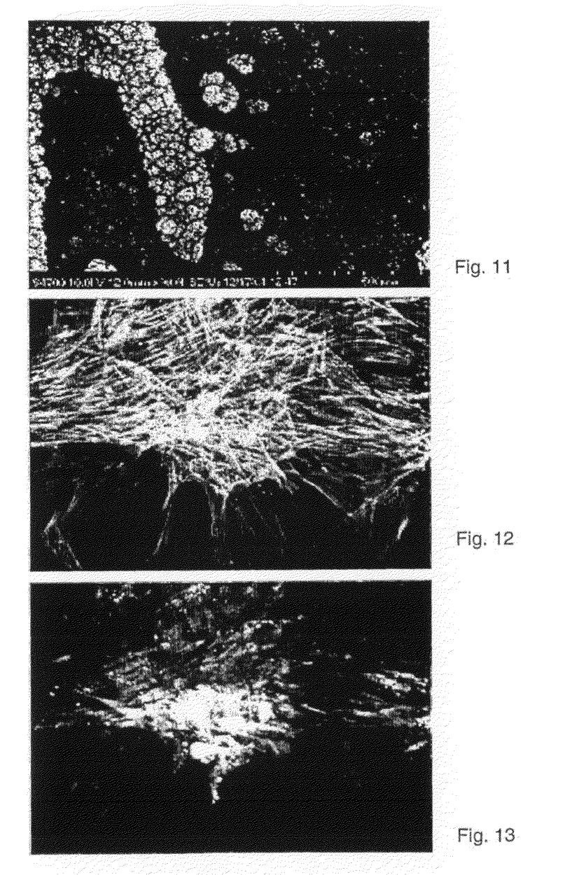

FIG. 11 shows an SEM image of filopodia of osteoprogenitor cells cultured on a substrate having near orthogonal (±50 nm) pits.

FIG. 12 shows actin staining of the substrate of FIG. 11.

FIG. 13 shown OPN staining of the substrate of FIG. 11.

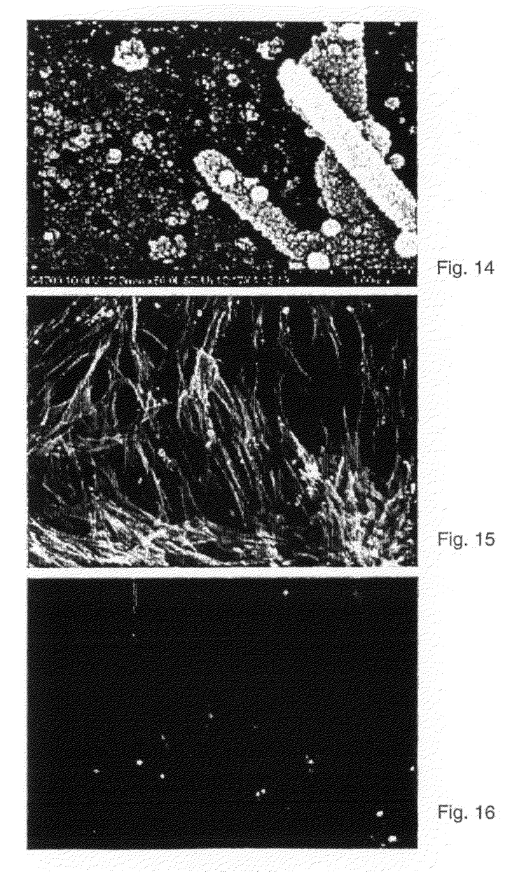

FIG. 14 shows an SEM image of filopodia of osteoprogenitor cells cultured on a substrate having hexagonal pits.

FIG. 15 shows actin staining of the substrate of FIG. 14.

FIG. 16 shows OPN staining of the substrate of FIG. 14.

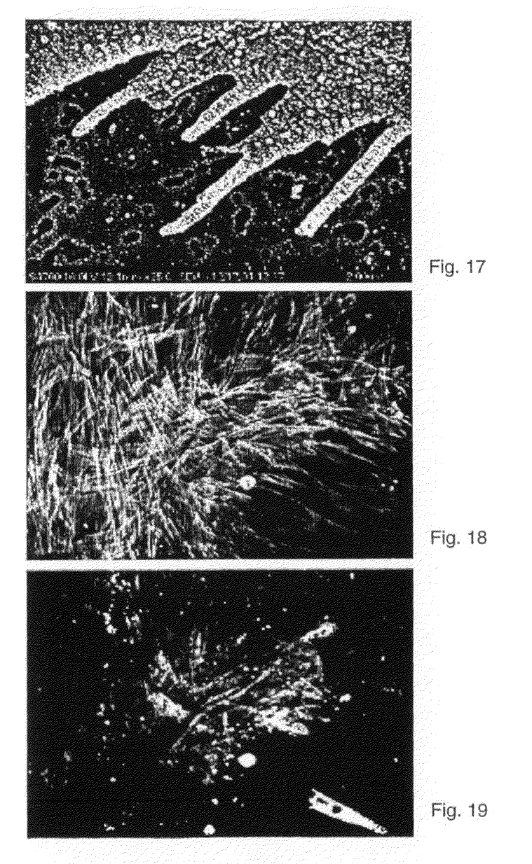

FIG. 17 shows an SEM image of filopodia of osteoprogenitor cells cultured on a substrate having random pits.

FIG. 18 shows actin staining of the substrate of FIG. 17.

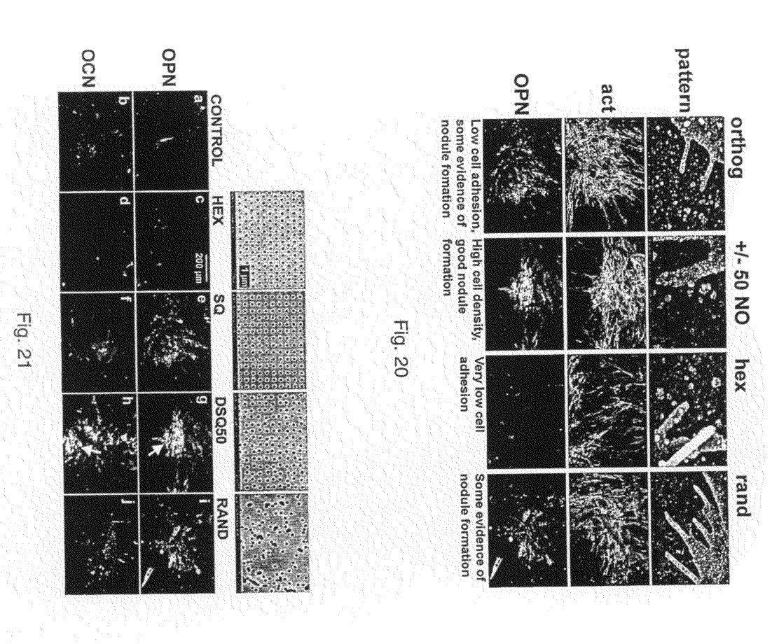

FIG. 19 shows OPN staining of the substrate of FIG. 17.

FIG. 20 shows a composite of the images of FIGS. 8-19, for ease of comparison.

FIG. 21 shows OPN staining (a, c, e, g and i) and OCN staining (b, d, f, h and j) for osteoprogenitor cells cultured on (a & b) a planar control substrate, (c & d) a substrate having hexagonal pits (HEX), (e & f) a substrate having a square array of pits (SQ), (g & h) a substrate having a disordered square array of pits (±50 nm) (DSQ50), (i & j) a substrate having random pits (RAND). The nano-patterns used are also shown.

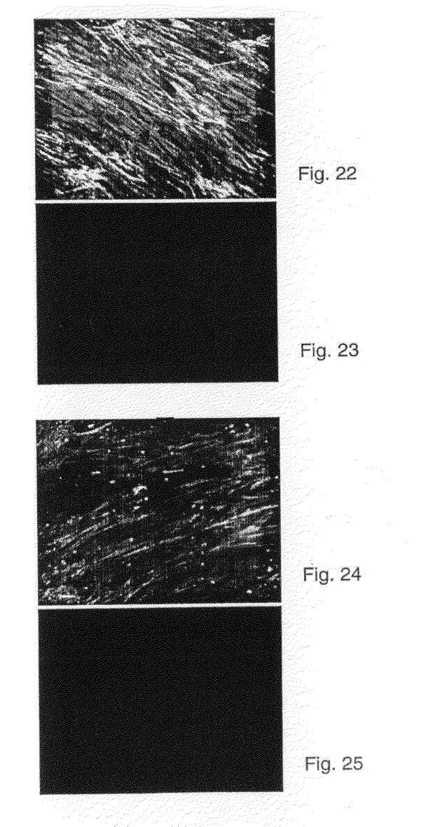

FIG. 22 shows actin staining for HMSCs cultured on a planar control substrate.

FIG. 23 shows OPN staining for HMSCs cultured on a planar control substrate.

FIG. 24 shows actin staining for HMSCs cultured on a substrate having orthogonal pits.

FIG. 25 shows OPN staining for HMSCs cultured on a substrate having orthogonal pits.

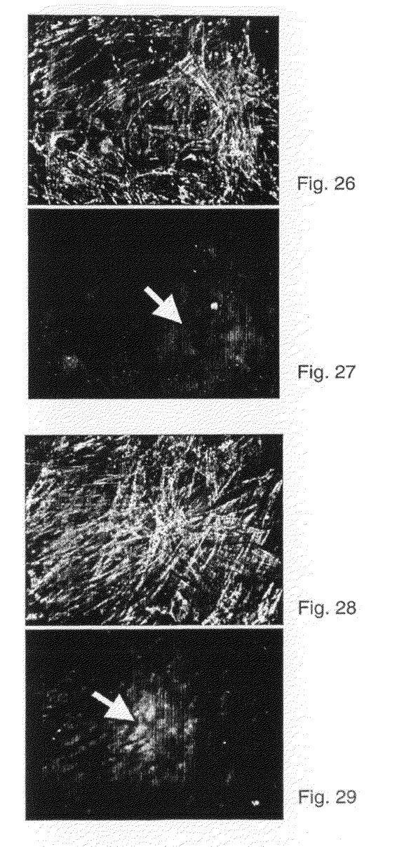

FIG. 26 shows actin staining for HMSCs cultured on a substrate having near orthogonal ±20 nm pits.

FIG. 27 shows OPN staining for HMSCs cultured on a substrate having near orthogonal ±20 nm pits.

FIG. 28 shows actin staining for HMSCs cultured on a substrate having near orthogonal ±50 nm pits.

FIG. 29 shows OPN staining for HMSCs cultured on a substrate having near orthogonal ±50 nm pits.

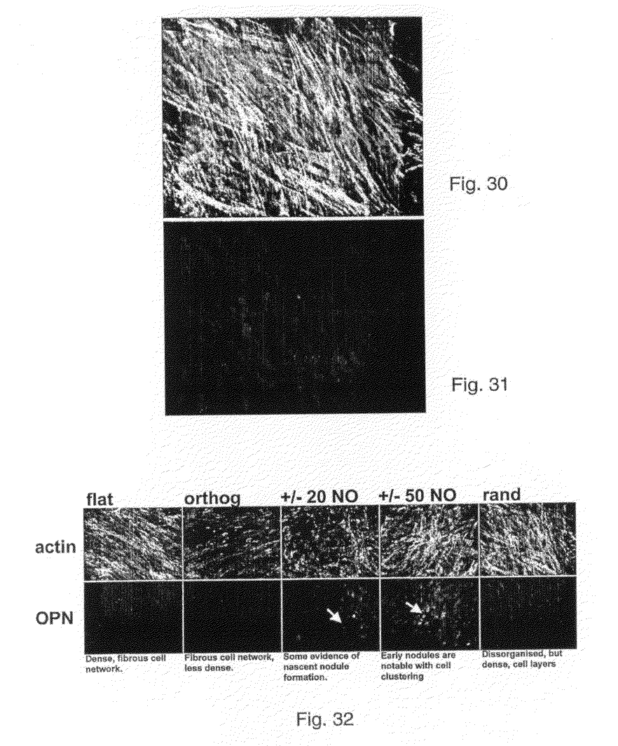

FIG. 30 shows actin staining for HMSCs cultured on a substrate having random pits.

FIG. 31 shows OPN staining for HMSCs cultured on a substrate having random pits.

FIG. 32 shows a composite of the images of FIGS. 22-31, for ease of comparison.

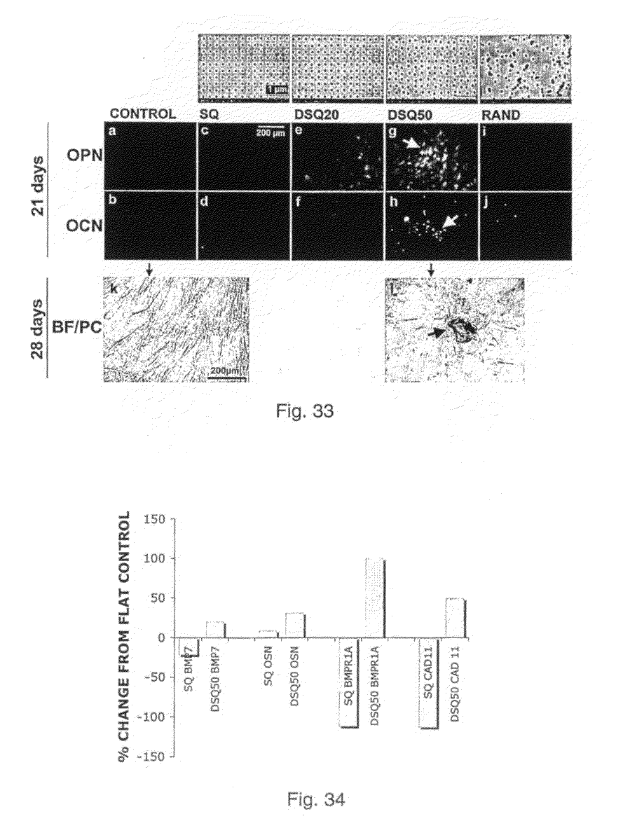

FIG. 33 shows OPN staining (a, c, e, g and i) and OCN staining (b, d, f, h and j) for HMSC cultured for 21 days on (a & b) a planar control substrate, (c & d) a substrate having a square array of pits (SQ), (e & f) a substrate having a disordered square array of pits (±20 nm) (DSQ20), (g & h) a substrate having a disordered square array of pits (±50 nm) (DSQ50), (i & j) a substrate having random pits (RAND). Bright field/phase contrast (BF/PC) images of alizarin red stained HMSCs cultured for 28 days on (k) a planar control substrate and (l) a substrate having a disordered square array of pits (±50 nm) are also shown. The scale bar shown on FIG. 33 k also applies to FIG. 331. The nano-patterns used for each substrate are shown above the figure.

FIG. 34 shows quantitative results from a cDNA microarray study for bone markers using HMSCs cultured for 21 days on substrates having either a square array of pits (SQ) or a disordered square array of pits (±50 nm) (DSQ50) compared to HMSCs cultured on a planar control substrate.

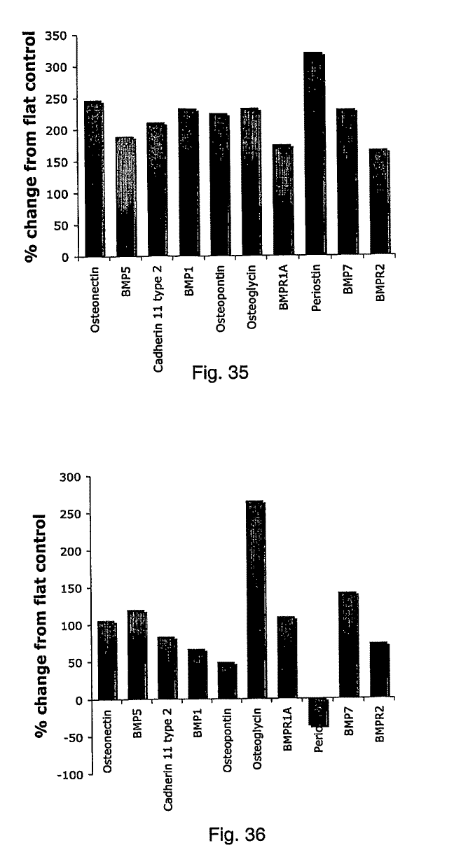

FIG. 35 shows microarray data for osteospecific genes using HMSCs cultured for 14 days on a substrate having a disordered square array of pits (±50 nm) compared to HMSCs cultured on a planar control substrate.

FIG. 36 shows microarray data for osteospecific genes using HMSCs cultured for 28 days on a substrate having a disordered square array of pits (±50 nm) compared to HMSCs cultured on a planar control substrate.

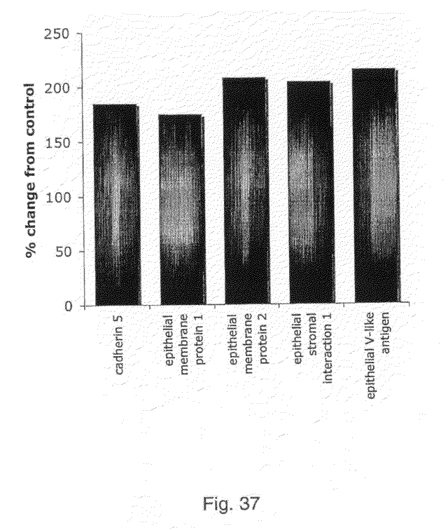

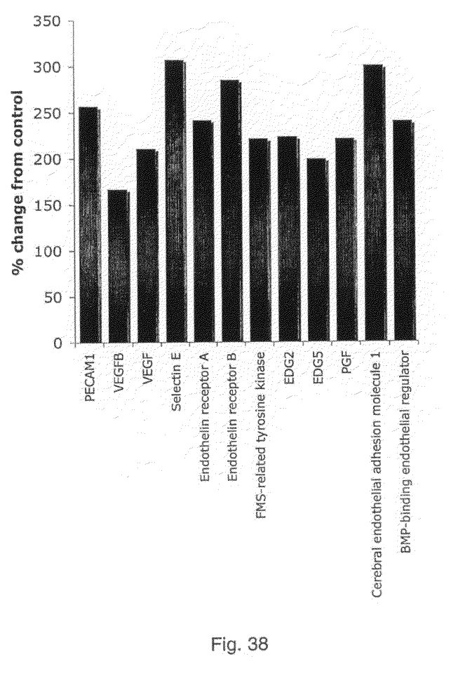

FIG. 37 shows microarray data for epithelial-related genes using HMSCs cultured for 14 days on a substrate having a disordered square array of pits (±50 nm) compared to HMSCs cultured on a planar control substrate.

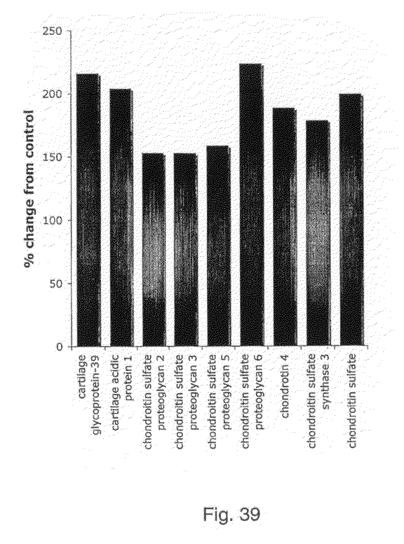

FIG. 38 shows microarray data for endothelial-related genes using HMSCs cultured for 14 days on a substrate having a disordered square array of pits (±50 nm) compared to HMSCs cultured on a planar control substrate.

FIG. 39 shows microarray data for cartilage-related genes using HMSCs cultured for 14 days on a substrate having a disordered square array of pits (±50 nm) compared to HMSCs cultured on a planar control substrate.

The specific embodiments of the invention are directed towards materials for bone implants. However, it is to be understood that the invention is not necessarily limited to this, since the inventors realise that, working within the scope of the invention, nanotopographical cues may be provided to stem cells to differentiate towards alternative functionalities.

First will be described the methods employed to manufacture biocompatible substrates according to embodiments of the invention, and alternative biocompatible substrates for comparison. Then will be described the particular experimental tests carried out on those substrates, with a discussion of the technical significance of the results.

### Manufacture of Biocompatible Substrates

In a preferred embodiment, a suitable pattern having a desired degree of mis-order is produced in a master. This master is formed of silicon in this embodiment, since patterning of silicon is well-understood. The silicon master is near atomically flat before patterning and is sufficiently conducting during the electron exposure to avoid sample charging. The desired pattern is generated by a computer program in which a suitable notional lattice is defined and each topographic feature is randomly displaced along the axes of the lattice by a random value. The software generates a file suitable for an electron beam lithography tool to read and execute. The silicon substrate is coated with a polymeric material, generally termed resist, which is susceptible to electron exposure. In the regions where the electron beam lithography tool exposes the resist, the regions will either be removed or left behind after development. This is determined by the type of resist used, generally termed positive or negative resist. Such considerations as the nature of the resist and the nature of the substrate will be well understood by a person skilled in the art.

Known suitable electron beam lithography tools have a grid resolution of 5 nm. Recently, more advanced electron beam lithography tools have become available that have a grid resolution of 1 nm. Suitable electron beam lithography tools will be known to persons skilled in the art. The resolution of the position of the topographic features is determined by the grid resolution of the electron beam lithography tool. However, there is also a stochastic displacement as a result of signal noise, temperature variations etc.

After patterning of the resist on the surface of the silicon, there are at least two options for forming a biocompatible polymeric substrate. For prototyping, the pattern formed in the resist can be transferred to the silicon through a reactive ion etch process. This yields a silicon surfaces with a topographic pattern which can be transferred by embossing to a suitable polymeric material. Alternatively, a nickel shim can be formed from the master structure by electro plating, a process well-known and used in the optical storage industry (CDs and DVDs). To make a nickel shim the master structure is first coated with a thin conducting metal film which subsequently acts as an electrode during the galvanic electroplating. The formed nickel shim is a negative copy of the master structure and can be used to make biocompatible replicas by embossing or injection moulding.

FIG. 1 shows a schematic plan view of a nanotopography 100 formed from nanopits 104, based on a notional square lattice (the notional lattice points being defined by the intersections of straight dashed lines). As shown in exaggerated form in this drawing, the nanopits 104 are offset from their respective notional lattice points. The maximum offset is shown in this case as C, defined by a dashed circle 102 of radius C surrounding each notional lattice point. However, it should be noted that the maximum offset need not be defined by a circle, but could be defined by a square (or rectangle) centred on each notional lattice point.

FIG. 2 shows a schematic plan view of a different nanotopography 200 formed from nanopits 204, based on a notional rectangular lattice (the notional lattice points being defined by the intersections of straight dashed lines).

FIG. 3 shows a schematic plan view of a different nanotopography 300 formed from nanopits 304, based on a notional hexagonal lattice (the notional lattice points being defined by the intersections of straight dashed lines).

In each of the schematic embodiments, the mis-order is apparent, but the arrangement of nanotopographical features is not truly random, due to the (statistically) relatively narrow distribution of distances between nearest neighbour topographical features. In other words, the arrangement of the topographical features does not allow for the creation of large gaps between features on the surface.

FIG. 4 shows an SEM micrograph of an orthogonal array of 120 nm diameter pits 100 nm deep with 300 nm centre to centre spacing.

FIG. 5 shows an SEM micrograph of an orthogonal array of 120 nm diameter pits 100 nm deep with 300 nm centre to centre spacing. Each pit has been randomly displaced from its grid position by +/−20 nm.

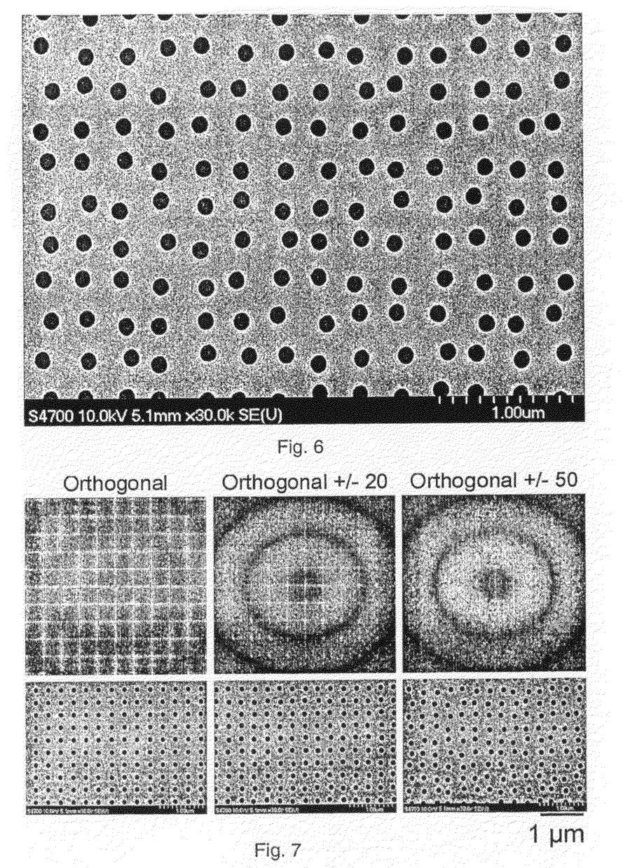

FIG. 6 shows an SEM micrograph of an orthogonal array of 120 nm diameter pits 100 nm deep with 300 nm centre to centre spacing. Each pit has been randomly displaced from its grid position by +/−50 nm.

FIG. 7 shows SEM micrographs of the orthogonal and nearly orthogonal nano pit arrays of FIGS. 4-6. The corresponding FFT images illustrate the decrease in long range order for the more disordered surface.

In the embodiments described herein, the degree of mis-order applied to a notional lattice uses the notation ±C, denoting that the maximum allowed deviation of each feature from its notional lattice point is a distance C along one axis of the lattice and a distance C along another axis of the lattice. For each feature, therefore, a deviation of between (and including) 0 and C is allowed, along each axis. Note that it is also possible to specify that the degree of mis-order along one axis is different to the degree of mis-order along another axis. Such asymmetrical mis-order would be denoted ±Cₐ and ±C_{b}, indicating the mis-order applied along axis a and the mis-order applied along axis b of the notional lattice.

Bone is characterised by a great potential for growth, regeneration and remodelling throughout life. This is largely due to the directed differentiation of mesenchymal stem cells into osteogenic cells, a process subject to exquisite regulation and a complex interplay by a variety of hormones, differentiation factors and environmental cues present within the bone matrix (Bianco et al 2001, Oreffo et al 2004, Oreffo et al 2005). In order to assess the effect of the prepared biocompatible substrates, the following tests were carried out.

### Effect of Surface Nantopography on Growth, Adhesion and Differentiation of Osteoprogenitor Cells

Initially, osteoprogenitor cells (pre-osteoblasts) were selected as a suitable cell type for testing the effect of selected nanotopographies on cell differentiation.

Bone marrow samples (female, n=4; mean 76+/−8 years of age) were obtained from hematologically normal patients undergoing routine hip replacement surgery. Only tissue that would have been discarded was used with the approval of the Southampton & South West Hants Local Research Ethics Committee. Primary cultures of bone marrow cells were established as previously described (Oreffo et al 1998).

Marrow aspirates were washed in α-MEM, then the suspended cells centrifuged at 250 g for 4 minutes at room temperature. The cell pellet was resuspended and plated to culture flasks at appropriate densities with non-adherent cells and red blood cells were removed via a PBS wash and media change after one week. Cultures were maintained in basal media (α-MEM containing 10% FCS) at 37° C., supplemented with 5% CO₂. All studies were conducted using passage 1 and passage 2 cells.

The osteoprogenitor cells were seeded onto the test materials at a density of 1×10⁴ cells per sample in 1 ml of complete medium. The medium used was α-MEM with 10% FCS (Life Technologies, UK). The cells were incubated at 37° C. with a 5% CO₂ atmosphere, and the medium was changed twice a week for 21 days.

At 21 days, the cells were formaldehyde fixed for fluorescence or gluteraldehyde fixed for SEM. For SEM, the cells were next post-fixed in Osmium tetroxide and dehydrated through a graded series of alcohols before air-drying with HMDS, gold coating and viewing. For fluorescence, cells were permeabilized with triton X and then dual stained with phalloidin-rhodamine to stain actin and antibodies for osteopontin (OPN) (an osteoblast specific marker protein). Secondary antibodies were then used to conjugate fluoroscein to the primary antibody.

In vitro, the differentiation of osteoprogenitor cells to mature, bone forming, osteoblasts is marked by the formation of bone nodules. These are sites where cells accumulate immediately before producing mineral (only osteoblastic cells will do this). These nodules act to protect the nascent mineral and also act as centres for the production of bone specific matrix (mainly collagen I, but specifically including OPN). The nascent mineral then acts as a nucleation site for crystal growth and the combination of mineral and matrix form the unique nature of bone (matrix giving ductility and strength, mineral giving hardness).

FIG. 8 shows filopodia of osteoprogenitor cells on orthogonal pits. Filopodia are the means by which cells locate nanoscale features. FIG. 9 shows actin staining allowing viewing of cell morphology. Here, cells can be seen to be starting to form a nodule, gathering in density in the centre of the image. FIG. 10 shows OPN staining. Towards the centre, slight rises in OPN intensity are observed.

FIG. 11 shows filopodial interaction with near orthogonal (±50 nm) pits. FIG. 12 shows actin staining and the cells can be seen to be accumulating in the centre of the image into a mature nodule. FIG. 13 shows OPN staining to be very intense, indicative of high levels of matrix production.

FIG. 14 shows filopodial interaction with hexagonal pits. FIG. 15 shows actin staining. At the bottom of the image is a planar area on which the cells can be seen to grow, the rest of the image is cells on the pits and very poor cell growth was observed. FIG. 16 shows that almost no OPN staining was observed on the topography.

FIG. 17 shows filopodial interaction with random pits. FIG. 18 shows actin staining of morphology and FIG. 19 OPN staining. Similar levels of nodule formation were observed as with the orthogonal pits.

FIG. 20 shows the composite image.

FIG. 21 shows staining for the osteoblast specific extracellular matrix protein osteocalcin (OCN), as well as OPN staining. Four different patterns were used: (i) a planar control substrate, (ii) a hexagonal array of pits with the distance between pits being 300 nm (HEX), (iii) a square array of pits on 300 nm centre-to-centre spacing (SQ), (iv) a disordered square array of pits, each pit displaced randomly by up to 50 nm on both axes from its position in a true square of 300 nm centre-to-centre spacing (DSQ50) and (v) a pattern of pits that were displaced randomly over a 150 by 150 μm field and this field repeated to fill the 1 cm² area (RAND). The pit diameter for all samples was 120 nm and the pit depth was 100 nm. On the planar control material, whilst good cell growth was observed, there was little evidence of osteoblast marker (OPN and OCN) production (FIG. 21 a and b). On the highly ordered symmetries, decrease in adhesion compared to the control was noted, especially on HEX, where very little osteoprogenitor cell growth was noted (FIG. 21 c-f). Osteoprogenitor cells on the random material grew well, but only slightly raised OPN or OCN levels were observed (FIG. 21 i&j). However, osteoprogenitor cells cultured on the DSQ50 nanotopography were seen to form bone nodule structures with high levels of OPN and OCN (FIG. 21 g&h).

### Effect of Surface Nantopography on Differentiation of Human Mesenchymal Stem Cells

Next, the fluorescence experiments were repeated with primary human mesenchymal stem cells (HMSCs). HMSCs can give rise to cells of the adipogenic (fat), chondrogenic (cartilage), osteoblastic (bone), myoblastic (muscle) and fibroblastic and reticular (connective tissue) lineages and generate intermediate progenitors with a degree of plasticity. Thus, HMSCs give rise to a hierarchy of bone cell populations with a number of developmental stages: MSC, determined osteoprogenitor cells, preosteoblast, osteoblast and ultimately, osteocyte. An ideal orthopaedic repair material would have to influence this osteoprogenitor cell mix in vivo to differentiate into mature osteoblasts, rather than connective tissue cell types.

HMSCs were isolated in a similar manner to the osteoprogenitor cells, however, very immature, purely stem cell populations were isolated by FACS with the stro-1 antibody. Again, the cells were cultured for 21 days.

FIG. 22 shows actin and FIG. 23 shows OPN for HMSCs cultured on planar control. Cell populations with typically fibroblastic appearance were noted with very low levels of OPN.

FIG. 24 shows actin and FIG. 25 shows OPN for HMSCs cultured on orthogonal pits. The cell appearance was similar, but less dense, to the cells on control.

FIG. 26 shows actin and FIG. 27 shows OPN for HMSCs cultured on near orthogonal ±20 nm pits. Nascent nodule formation was observed (arrows).

FIG. 28 shows actin and FIG. 29 shows OPN for HMSCs cultured on near orthogonal ±50 nm pits. Increased nodule formation was observed (arrows).

FIG. 30 shows actin and FIG. 31 shows OPN for HMSCs cultured on random pits. No nodule formation was observed.

FIG. 33 shows staining for osteoblastic ECM proteins OPN and OCN for HMSCs cultured on (i) a planar control substrate, (ii) a square array of pits on 300 nm centre-to-centre spacing (SQ), (iii) a disordered square array of pits, each pit displaced randomly by up to 20 nm on both axes from its position in a true square of 300 nm centre-to-centre spacing (DSQ20), (iv) a disordered square array of pits, each pit displaced randomly by up to 50 nm on both axes from its position in a true square of 300 nm centre-to-centre spacing (DSQ50) and (v) a pattern of pits that were displaced randomly over a 150 by 150 μm field and this field repeated to fill the 1 cm² area (RAND). On the planar control and SQ materials, the cells were fibroblastic in appearance, with highly elongated and aligned morphology, as opposed to the typical appearance of osteoblasts. Negligible OPN or OCN staining was observed (FIG. 33 a-d). HMSCs cultured on RAND for 21 days had a more osteoblastic morphology, but negligible OPN or OCN positive areas were observed (FIG. 33 i&j). Again, cells on DSQ20 had a more osteoblastic morphology and expressed intense foci of OPN, however, negligible OCN was noted (FIG. 33 e&f). HMSCs cultured on DSQ50 showed signs of early nodule formation and displayed both OPN and OCN positive areas (FIG. 33 g&h). The comparison of results is interesting as it shows that the HMSCs, an enriched stem cell population, can form both purely fibroblastic and predominantly osteoblastic populations by simply adding a discrete level of disorganization.

Another batch of HMSCs was cultured for 28 days on each of the nanotopographies before alizarin red staining of bone mineral (FIG. 33 k&l). This extended culture time of 28 days allowed positive identification of mineralization within nodules (FIG. 331). This was only noted in HMSCs cultured on the DSQ50 topography. HMSCs cultured on the control for 28 days had a fibroblastic morphology. It is important to note that no osteogenic supplements (e.g. dexamethasone, ascorbate) were used in these experiments. Therefore, nanoscale disorder can stimulate stem cells to form nodules and to produce bone mineral in vitro in basal media (αMEM with 10% FCS).

It should be noted that the slower development of nodules with the HMSCs was to be expected as they are enriched in stem cells and are therefore less differentiated and committed in general than the mixed population osteoprogenitor cells. Thus, it was also not surprising that less OCN and OPN positive stain was observed.

Changes in Expression of Osteospecific Genes in HMSCs Cultured on Substrates with Different Surface Nanotopographies

Quantitative results derived from a cDNA microarray study for the bone markers bone morphogenic protein 7 (BMP7), osteonectin (OSN), bone morphogenetic protein receptor, type IA precursor (BMPR1A) and cadherin 11 (CAD1) after 21 days of HMSC culture demonstrated that the low adhesion SQ topography suppressed markers of osteoblast activity, whereas the DSQ50 increased transcription of genes related to osteogenesis (FIG. 34). BMP7 is involved in early stage bone formation, OSN is a bone specific extracellular matrix protein (as are OPN and OCN), BMPR1A is a marker of bone stem cell activity and CAD11 is expressed during bone development and maintenance (information on genes derived from Stanford Source, see Diehn et al, 2003). The increased expression of such genes indicates that after 21 days the HMSCs were preparing to form mineralized matrix, as shown by alizarin red staining at 28 days.

In order to elucidate further the extent of DSQ50's osteogenic ability, small osteospecific oligoarrays were used with HMSCs cultured for 14 days on control and DSQ50 substrates. Day 14 was selected as it is the time point at which the cells would start to differentiate (i.e. once proliferation has slowed) (Stein et al, 1993), thus a true assessment of the extent of osteoblast activation from HMSCs could be obtained. None of the 101 osteospecific genes studied was expressed in HMSCs cultured on a planar control for 14 days. However, expression of thirteen osteospecific genes was observed in HMSCs cultured on DSQ50. These genes are listed in Table 1.

These expressed genes were in the areas of cell signalling, bone specific extracellular matrix production (notably OCN) and calcium and phosphate control (required for bone mineral formation) (Diehn et al, 2003).

TABLE 1

| Gene | Function |
|---|---|
| Transforming | Growth factor that influences |
| growth factor β | osteoblast differentiation. |
| receptor 1 | |
| (TGFBR1). | |
| Transforming | Growth factor that influences |
| growth factor β 3. | osteoblast differentiation. |
| Matrix | Remodelling of type I, II and III |
| metallopeptidase | collagens. |
| 8 (MMP 8). | |
| Integrin α1. | Involved in cell-matrix adhesion. |
| Integrin, alpha M | Involved in cell-matrix adhesion. |
| (complement | |
| component 3 | |
| receptor 3 | |
| subunit) (ITGAM). | |
| Intercellular | Involved in cell-cell adhesion. |
| adhesion molecule | |
| 1 (CD54) (ICAM1). | |
| Colony | Involved in osteospecific |
| stimulating | differentiation (interacts with Cbfa1, |
| factor 2 (CSF 2). | an osteospecific transcription factor). |
| Collagen 5A1. | A bone phenotype collagen. Involved in |
| | phosphate transport. |
| Collagen 7A1. | A bone phenotype collagen. Involved in |
| | phosphate transport. |
| Collagen 1A1. | The main bone collagen. |
| Osteocalcin | Bone specific extracellular matrix |
| (BGLAP). | protein. Required for mineralization. |
| Arylsulfatase E | Calcium ion binding. Required for |
| (ARSE). | correct composition of bone matrix. |
| Alkaline | Regulates phosphate levels. Required |
| phosphatase | for ossification. |
| (ALPL). | |

The HMSCs used in the following experiments were obtained from a different patient from the HMSCs used in the experiments described above.

FIG. 35 shows microarray data obtained using RNA extracted from HMSCs cultured on DSQ50 for 14 days. These data showed that expression of each of the osteospecific genes osteonectin, BMP5, cadherin 11 type 2, BMP1, osteopontin, osteoglycin, BMPR1A, periostin, BMP7 and BMPR2 was up-regulated by at least 100% than for cells cultured on a flat control substrate. At 14 days, cell proliferation would have been slowing down and osteospecific genes would be expected to be switched on in preparation for protein production and mineralization.

FIG. 36 shows microarray data obtained using RNA extracted from HMSCs cultured on DSQ50 for 28 days. These data showed that although the cells are more osteoblastic than cells cultured on control substrate, expression of each of the osteospecific genes osteonectin, BMP5, cadherin 11 type 2, BMP1, osteopontin, osteoglycin, BMPR1A, periostin, BMP7 and BMPR2 was, in many cases, less up-regulated than at 14 days. At 28 days, the gene changes would have been translated into changes in the proteome and therefore the gene changes are not as important. Also, after 28 days, the cells on the flat controls will probably be starting to produce some osteospecific gene changes, as seen for DSQ 50, but after a much increased time period.

These results clearly show that maintaining feature size, but altering symmetry and disorder can make surfaces that have almost no stem cell adhesion support high levels of stem cell differentiation.

Differentiation of Stem Cells Cultured on Substrates with Different Surface Nanotopographies

Microarray analysis using RNA extracted from HMSCs cultured on DSQ50 for 14 days was performed to determine whether there was general stem cell activation when cells were grown on this substrate. FIG. 37 shows that the expression of each of the epithelial-related genes cadherin 5, epithelial membrane protein 1, epithelial membrane protein 2, epithelial stromal interaction 1 and epithelial V-like antigen was up-regulated by at least 100% compared to expression of these genes in cells cultured on a planar control. FIG. 38 shows that expression of each of the endothelial-related genes platelet/endothelial cell adhesion molecule (PECAM), vascular endothelial growth factor-B (VEGFB), VEGF, selectin E, endothelin receptor receptor A, endothelin receptor B, FMS-related tyrosine kinase, endothelial differentiation lysophosphatidic acid G-protein-coupled receptor-2 (EDG2), EDG5, PGF, cerebral endothelial adhesion molecule 1 and BMP-binding endothelial regulator was up-regulated by at least 100% compared to expression of these genes in cells cultured on a planar control. FIG. 39 shows that expression of each of the cartilage-related proteins cartilage glycoprotein-39, cartilage acidic protein 1, chondroitin sulphate proteoglycan 2, chondroitin sulphate proteoglycan 3, chondroitin sulphate proteoglycan 5, chondroitin sulphate proteoglycan 6, chondroitin 4, chondroitin sulphate synthase 3 and chondroitin sulphate was up-regulated by at least 100% compared to expression of these genes in cells cultured on a planar control.

Therefore, there was general stem cell activation for cells grown on the DSQ50 nanotopography.

Modifications to these embodiments, further embodiments and modifications thereof will be apparent to the skilled person on reading this disclosure, and as such are within the scope of this invention. In particular, although the embodiments described above are for flat substrates, it will be apparent that the invention may also be applied to curved substrates, or substrates with irregular surfaces.

### REFERENCES

Charnley J. Surgery of the hip-joint: present and future developments. Br Med J 1960; 5176:821-6.
Charnley J. Anchorage of the femoral head prosthesis to the shaft of the femur. J Bone Joint Surg Br 1960; 42-B:28-30.
France R, Haddow D. Biomaterials to Order. Chemistry in Britain 2000:29-31.
Freeman M A, Bradley G W, Revell P A. Observations upon the interface between bone and polymethylmethacrylate cement. J Bone Joint Surg Br 1982; 64(4):489-93.
Gefen A. Computational simulations of stress shielding and bone resorption around existing and computer-designed orthopaedic screws. Med Biol Eng Comput 2002; 40(3):311-22.
Dalby M J, Riehle M O, Johnstone H, Affrossman S, Curtis A S. Investigating the limits of filopodial sensing: a brief report using SEM to image the interaction between 10 nm high nano-topography and fibroblast filopodia. Cell Biol Int 2004; 28(3):229-36.
Dalby M J, Riehle M O, Johnstone H, Affrossman S, Curtis A S. In vitro reaction of endothelial cells to polymer demixed nanotopography. Biomaterials 2002; 23(14):2945-54.
Gallagher J O, McGhee K F, Wilkinson C D W, Riehle M O. Interaction of Animal Cells with Ordered Nanotopography. IEEE Transactions on Nanobioscience 2002; 1(1):24-28.
Berry C C, Campbell G, Spadiccino A, Robertson M, Curtis A S. The influence of microscale topography on fibroblast attachment and motility. Biomaterials 2004; 25(26):5781-8.
Dalby M J, Riehle M O, Johnstone H J, Affrossman S, Curtis A S. Polymer-Demixed Nanotopography: Control of Fibroblast Spreading and Proliferation. Tissue Eng 2002; 8(6):1099-1108.
Dalby M J, Berry C C, Riehle M O, Sutherland D S, Agheli H, Curtis A S. Attempted endocytosis of nano-environment produced by colloidal lithography by human fibroblasts. Exp Cell Res 2004; 295(2):387-94.
Dalby M J, Yarwood S J, Johnstone H, Affrossman S, Riehle M. Fibroblast Signalling Events in response to Nanotopography: A Gene Array Study. IEEE Transactions on Nanobioscience 2002; 1(1):12-17.
Dalby M J, Yarwood S J, Riehle M O, Johnstone H J, Affrossman S, Curtis A S. Increasing fibroblast response to materials using nanotopography: morphological and genetic measurements of cell response to 13-nm-high polymer demixed islands. Exp Cell Res 2002; 276(1):1-9.
Price R L, Haberstroh K M, Webster T J. Enhanced functions of osteoblasts on nanostructured surfaces of carbon and alumina. Med Biol Eng Comput 2003; 41(3):372-5.
Webster T J, Ergun C, Doremus R H, Siegel R W, Bizios R. Enhanced osteoclast-like cell functions on nanophase ceramics. Biomaterials 2001; 22(11):1327-33.
Thapa A, Webster T J, Haberstroh K M. Polymers with nano-dimensional surface features enhance bladder smooth muscle cell adhesion. J Biomed Mater Res 2003; 67A(4):1374-83.
Andersson A S, Backhed F, von Euler A, Richter-Dahlfors A, Sutherland D, Kasemo B. Nanoscale features influence epithelial cell morphology and cytokine production. Biomaterials 2003; 24(20):3427-36.
Andersson A S, Brink J, Lidberg U, Sutherland D S. Influence of Systematically Varied nanoscale Topography on the Morhphology of Epithelial Cells. IEEE Transactions on Nanobioscience 2003; 2(2):49-57.
Sutherland D S, Broberg M, Nygren H, Kasemo B. Influence of Nanoscale Surface Topography on the Functional Behaviour of an Absorbed Model Macromolecule. Macromolecular Bioscience 2001; 1(6):270-273.
Denis F A, Hanarp P, Sutherland D S, Gold J, Mustin C, Rouxhet P G, et al. Protein Adsorption on Model Surfaces with Controlled Nanotopography and Chemistry. Langmuir 2002; 18(3):819-828.
Gadegaard N, Thoms S, MacIntyre D S, McGhee K, Gallagher J, Casey B, et al. Arrays of Nano-Dots for Cellular Engineering. Microelectronic Engineering 2003; 67-68:162-168.
Wilkinson C D W, Riehle M, Wood M, Gallagher J, Curtis A S G. The Use of Materials Patterned on a Nano- and Micro-Metric Scale in Cellular Engineering. Materials Science and Engineering. 2002; 19:263-269.
Curtis A S, Casey B, Gallagher J O, Pasqui D, Wood M A, Wilkinson C D. Substratum nanotopography and the adhesion of biological cells. Are symmetry or regularity of nanotopography important? Biophys Chem 2001; 94(3):275-83.
Cumming D R S, Thoms S, Beaumont S P, Weaver J M R. Fabrication of 3 nm wires using 100 keV electron beam lithography and poly(methyl methacrylate) resist. Applied Phys. Letters 1996; 68:322-324.
Vieu C, Carcenac F, Pepin A, Chen Y, Mejias M, Lebib A, et al. Electron Beam Lithography: Resolution Limits and Applications. Applied Surface Science 2000; 164:111-117.
Bianco P, Robey P G. Stem Cells in Tissue Engineering. Nature 2001; 414:118-121.
Oreffo R O C. Growth Factors for Skeletal Reconstruction and Fracture Repair. Curr. Opin. Investig. Drugs 2004; 5(4):419-423.
Oreffo R O C, Cooper C, Mason C, Clements M. Mesenchymal Stem Cells: Lineage, Plasticity and Skeletal Therapeutic Potential. Stem Cell Reviews 2005 (In Press).
Oreffo R O, Bord S, Triffitt J T. Skeletal progenitor cells and ageing human populations. Clin Sci (Lond) 1998; 94(5):549-55.
Diehn M, Sherlock G, Binkley G, Jin H, Matese J C, Hernandez-Boussard T, Rees C A, Chemy J M, Botstein D, Brown P O, Alizadeh A A. SOURCE: a unified genomic resource of functional annotations, ontologies, and gene expression data. Nucleic Acids Research 2003; 31(1):219-223.
Stein G S, Lian J B. Molecular Mechanisms Mediating Developmental and Hormone-regulated Expression of Genes in Osteoblasts. Ed. Academic Press: New York, 1993; page 47-91.

1. A biocompatible substrate for cell adhesion, differentiation, culture and/or growth, the substrate having an arrangement of topographical features arrayed in a pattern based on a notional symmetrical lattice in which the distance between nearest neighbour notional lattice points is C and is between 10 nm and 10 μm, and wherein the topographical features are locally mis-ordered such that the centre of each topographical feature is a distance of up to one half of C from its respective notional lattice point.

2. A substrate according to claim 1 wherein the topographical features of the biocompatible substrate are recesses into and/or protrusions from the surface of the substrate.

3. A substrate according to claim 1 wherein C is at least 100 nm.

4. substrate according to claim 1 wherein C is at most 3 μm.

5. A substrate according to claim 1 wherein the height or depth of the topographical features is at least 5% of C from the remainder of the surface of the substrate.

6. A substrate according to claim 1 wherein each topographical feature has substantially the same shape.

7. A substrate according to claim 1 wherein the diameter of the topographical features is at least 20 nm.

8. A substrate according to claim 1 wherein the centre of each topographical feature is at most one third of C from its respective notional lattice point.

9. A substrate according to claim 1 wherein the nature of the symmetry on which the notional lattice is based is selected from a parallelogram lattice, a rectangular lattice, a square lattice, a rhombic lattice, a trigonal lattice and a hexagonal lattice.

10. A method of manufacturing a biocompatible substrate for cell adhesion, differentiation, culture and/or growth, the substrate having an arrangement of topographical features arrayed in a pattern based on a notional symmetrical lattice in which the distance between nearest neighbour notional lattice points is C and is between 10 nm and 10 μm, and wherein the topographical feature is a distance of up to one half of C from its respective notional lattice point, the method including the steps of designing the notional symmetrical lattice, applying a degree of mis-order to the notional symmetrical lattice by requiring that the centre of each topographical feature is up to one third of C from its respective notional lattice point, thereby designing a mis-ordered lattice, and manufacturing the substrate according to the mis-ordered lattice.

11. A method according to claim 10 wherein the degree of mis-order is applied to each notional lattice point by a calculation step in which a random number is generated and used to provide one or more displacement amounts to said notional lattice point.

12. A method according to claim 10 wherein the method comprises the step of forming an array of topographical features using electron beam lithography on the surface of a master substrate.

13. A method according to claim 12 wherein the master substrate is used to create an intermediate substrate, the intermediate substrate then being used to form the final substrate.

14. A method for promoting differentiation of cells using a biocompatible substrate having an arrangement of topographical features arrayed in a pattern based on a notional symmetrical lattice in which the distance between nearest neighbour notional lattice points is C and is between 10 nm and 10 μm, and wherein the topographical features are locally mis-ordered such that the centre of each topographical feature is a distance of up to one half of C from its respective notional lattice point, in which method a population of said cells is located on said substrate for interaction with said arrangement of topographical features.

15. A method according to claim 14, wherein the cells are osteoprogenitor cells or mesenchymal stem cells.

16. A method according to claim 15, wherein the osteoprogenitor cells differentiate into osteoblasts and remain adhered to the substrate.

17. A bone repair prosthesis having a surface with an arrangement of topographical features arrayed in a pattern based on a notional symmetrical lattice in which the distance between nearest neighbour notional lattice points is C and is between 10 nm and 10 μm, and wherein the topographical features are locally mis-ordered such that the centre of each topographical feature is a distance of up to one half of C from its respective notional lattice point.
